(19) United States
(12) Patent Application Publication
(10) Pub. No.: US 2015/0087004 A1
(43) Pub. Date: Mar. 26, 2015

(54) Microfabricated 3D Cell Culture System

(71) Applicant: The Trustees of the University of Pennsylvania, Philadelphia, PA (US)

(72) Inventors: Christopher S. Chen, Princeton, NJ (US); Duc-Huy T. Nguyen, Philadelphia, PA (US); Sarah C. Stapleton, Philadelphia, PA (US); Michael T. Yang, Philadelphia, PA (US)

(73) Assignee: The Trustees of the University of Pennsylvania, Philadelphia, PA (US)

(21) Appl. No.: 14/334,408

(22) Filed: Jul. 17, 2014

Related U.S. Application Data
(63) Continuation of application No. PCT/US13/24450, filed on Feb. 1, 2013.
(60) Provisional application No. 61/594,084, filed on Feb. 2, 2012.

(51) Int. Cl. C12N 5/00; B29C 65/70; B29D 22/00; G01N 33/50

(52) U.S. Cl. CPC C12N 5/0068; G01N 33/5023; B29C 65/70; B29D 22/00; C12N 2535/00
USPC 435/29; 435/402; 156/245

(57) ABSTRACT

Device for 3D cell culture using an extracellular matrix including a substrate having at least one interior chamber, at least one opening providing access to the interior chamber for introduction of an extracellular matrix, and at least one channel disposed through at least a portion of the extra cellular matrix.

### CROSS-REFERENCE TO RELATED APPLICATION

This application is a U.S. continuation of PCT/U.S. Ser. No. 13/024,450, filed Feb. 1, 2013, which claims priority to U.S. Provisional Application Ser. No. 61/594,084, filed Feb. 2, 2012, and both of which are incorporated herein by reference in their entireties.

### STATEMENT REGARDING FEDERALLY SPONSORED RESEARCH OR DEVELOPMENT

This invention was made with government support under Grant No. EB00262, awarded by the National Institute of Health. The government has certain rights in the invention.

### BACKGROUND

The presently disclosed subject matter relates to techniques for culturing tissue, including by using a microfabricated 3D cell culture system.

The architecture of tissues can directly impact cellular and multicellular function. Within the in vivo microenvironment, cells adhere to the surrounding extracellular matrix (ECM), which has compositional, mechanical and topographic properties that are tissue specific. Cells can also be exposed to complex spatial distributions of soluble factors and can engage in physical interactions with neighboring cells. When cells are removed from this setting and cultured in vitro, typically on tissue culture polystyrene, they can rapidly lose their native phenotype. Cellular functions such as proliferation, migration and differentiation can be drastically altered in vitro. Certain in vitro studies have offered limited insight into the mechanisms governing biological processes.

One alternative to culturing cells in vitro is to observe them in vivo. When used in conjunction with genetic knockout methods and intravital microscopy methods, animal models such as zebrafish, drosophila and transgenic mice can be very informative, particularly in studies of organism development. However, in vivo assays generally can be more expensive, lower throughput, and harder to manipulate than in vitro assays. As such, they are often not employed as extensively in more commercial-driven applications such as drug discovery.

In addition to conventional in vitro cell culture and animal models, in vitro organotypic cultures can recapitulate some of the elements of in vivo tissue architecture. For example, breast cancer can be modeled in vitro by culturing a mass of epithelial cells within Matrigel, a mouse-derived mixture of ECM proteins. The cells can subsequently organize into hollow spherical structures known as mammary acini. As another example, the granulation tissue that forms during wound healing can be modeled as a polymerized matrix of collagen I in which fibroblasts can be embedded. The fibroblasts can subsequently contract the collagen matrix, yielding a dermal equivalent. One limitation of these organotypic models is that they are essentially self-assembled processes within a microenvironment whose composition is still poorly defined. To partially address some of these limitations, bioengineering approaches can be used, particularly in a 2D environment. For example, synthetic ECMs with tunable stiffness, adhesiveness and bioactivity can be engineered. Microfabrication-based approaches can be used to specify adhesive topography, modulate substrate stiffness, generate gradients soluble factor, and constrain cell-cell interactions.

One aspect of these culture models is that nearly all tissues have fluid-filled tubes that are lined with cells and surrounded by interstitial extracellular matrix containing a mixture of cells. This tissue architecture is true of glandular tissues such as pancreas (epithelialized tubes are filled with digestive enzymes), breast (lactiferous ducts carry milk), liver (bile ducts carry bile), brain (ventricles contain cerebrospinal fluid), intestines (transports food) and kidney (renal tubules carry waste filtrate). It is also true of all tissues fed by the circulatory system, as blood passes through an endothelial cell-lined vascular tree that permeates nearly all tissues in the body of all species with a closed circulatory system (including humans).

Accordingly, there remains a desire for improved in vitro environments for 3D cell culture.

### SUMMARY

The presently disclosed subject matter relates to devices for culturing tissue, particularly to systems and methods of a microfabricated 3D cell culture system including fluidic and matrix compartments.

According to some embodiments, a device for 3D cell culture includes a substrate having at least one interior chamber, at least one opening providing access to the at least one chamber for introduction of an extracellular matrix into the chamber, and at least one channel through the extracellular matrix. In some embodiments, the device for 3D cell culture can include at least one reservoir in fluid communication with the at least one channel.

In one embodiment, the device for 3D cell culture can include a plurality of channels through the extracellular matrix. Each channel can be connected, in fluid communication, to at least one reservoir for introduction of liquids, including but not limited to culture media, biological fluids (e.g., blood or its components, urine, milk, mucus, gastrointestinal fluids, or bile), and environmental fluids (e.g., seawater or lake water) to the channel. In one embodiment, each channel can be connected, in fluid communication, to two reservoirs.

In another embodiment, a method of fabricating a device for 3D cell culture includes providing a top master mold for fabricating a top layer. The mold can have raised portions defining at least one opening for introduction of extracellular matrix and at least one reservoir for introduction of media. A top layer can be cast from the master mold. A bottom master mold can be provided for fabricating a bottom layer. The bottom master mold can have raised portions defining an interior chamber and reservoirs for introduction of media. A bottom layer can be cast from the bottom master mold. The top and bottom layers can be treated to adhere the layers together.

In one embodiment, the top and bottom layers can form a microfabricated gap between the layers for introduction of an acupuncture needle into the internal chamber. The extracellular matrix can then be introduced, form a gel, and the needle can be removed.

In other embodiments, methods of using the device disclosed herein are provided. In one embodiment, the device can be used for introducing endothelial cells into a first channel and angiogenic factors into a second channel, creating a gradient of angiogenic factors extending across the extracellular matrix. In other embodiments, tumor cells can be introduced into either channel or the extracellular matrix while the first channel is endothelialized. In yet other embodiments, immune cells and fibroblasts can be introduced into either the channels or the extracellular matrix.

### BRIEF DESCRIPTION OF THE DRAWINGS

FIG. 1A-D is a schematic of the fabrication process of one embodiment of the presently disclosed subject matter.

FIG. 2A-D is a depiction of endothelial cell sprouting and vessel development in a microfabricated 3D cell culture device according to one embodiment of the presently disclosed subject matter.

FIG. 3 is a schematic of a multiple-well format of one embodiment of the presently disclosed subject matter.

FIG. 4A-B is a diagram illustrating applications of one embodiment of the presently disclosed subject matter.

FIG. 5A-F is a diagram illustrating additional applications of embodiments of the presently disclosed subject matter.

FIG. 6A-B is a diagram illustrating yet additional applications of embodiments of the presently disclosed subject matter.

FIG. 7A-I is a diagram illustrating different embodiments of the disclosed subject matter.

FIG. 8A-B is a diagram illustrating additional embodiments of the disclosed subject matter and applications thereof.

FIG. 9 is a flow diagram illustrating a method of fabricating a device for 3D culturing according to one embodiment of the disclosed subject matter.

FIG. 10 is a flow diagram illustrating a method of using the device for 3D culturing disclosed herein according to one embodiment of the disclosed subject matter.

FIG. 11A is schematic diagram of a device in accordance with an exemplary embodiment of the disclosed subject matter.

FIG. 11B is a photograph of a device in accordance with an exemplary embodiment of the disclosed subject matter.

FIG. 11C depicts a representative confocal immunofluorescence image of sprouting and migrating endothelial cells in accordance with an example of the disclosed subject matter.

FIG. 11D depicts a merged image of a time-lapse movie, and corresponding confocal image, tracking the position of fluorescent beads perfused through channels of an exemplary device in accordance with an example of the disclosed subject matter.

FIG. 12A-K shows representative confocal immunofluorescence images of sprouts and neovessels in accordance with an example of the disclosed subject matter.

FIG. 13A-F illustrates the effects of VEGFR2 inhibition on angiogenic sprouting in accordance with an example of the disclosed subject matter.

FIG. 14A-F illustrates the effects of S1P receptor inhibition on angiogenic sprouting in accordance with an example of the disclosed subject matter.

FIG. 15 is a plot of sprout length and the number of sprout tip cells and single cells after four days of exposure to various factors in accordance with an example of the disclosed subject matter.

### DETAILED DESCRIPTION

The disclosed subject matter is generally directed to techniques for culturing tissue in a microfabricated 3D cell culture system. Reference will now be made in detail to embodiments of the disclosed subject matter, examples of which are illustrated in the accompanying drawings.

Throughout this specification, the term “tubular compartment(s),” “channel,” “sink channel,” and “source channel” shall mean a cavity through an extracellular matrix. The terms “tube,” “tubular compartment,” and “channel” shall be synonymous with each other unless the context indicates otherwise. It shall be appreciated that a “tubular compartment” is not required to have a circular or elliptical cross section, but could instead take on other shapes.

Likewise, the term “interstitial” region shall mean the region of the extracellular matrix adjacent to or between one or more channels. The term “extracellular matrix” or “ECM” can be used to refer to any material that provides structural support for the channels. The ECM can have a synthetic composition (e.g., polyethylene glycol, poly(lactic-co-glycolic acid)) or be derived from natural components such as proteins (e.g., collagen, fibrin) or polysaccharides (e.g., alginate, agarose, dextran), or living cells themselves and their byproducts.

In one aspect of the disclosed subject matter, a device for 3D cell culture includes a substrate having at least one interior chamber. The substrate includes an opening that provides access to the interior chamber(s) for introduction of an extracellular matrix. The extracellular matrix has at least one channel defined therethrough.

In one embodiment, a device for 3D cell culture comprises one or more tubular channels that are encased within extracellular matrix representing the interstitial region. In some embodiments, the channels can be connected to fluidic reservoirs. The tubular channels can be individually addressable compartments that can be seeded separately with any cell type and perfused with any type of fluid. For example, for a device having two channels, both compartments could be lined with different cells and perfused with different media. The surrounding ECM can be comprised of any type of native or synthetic ECM or filled with any cell type. Moreover, this device can be re-configured in a number of ways, including altering the interactions, number, geometry, and orientation of tubular compartments. In certain embodiments, for example, the tubular channels can be interconnected.

As disclosed herein, the tubular compartments can contain more than one concentric layer of different ECMs or other biocompatible and biodegradable coating materials that can be seeded with different cells. For example, and not limitation, the tubular compartments can contain an endothelium, intima layer, media layer (e.g., where smooth muscle cells are located), and aventitia layer associated with a blood vessel.

The techniques described herein can provide for improved in vitro environments for 3D cell culture not limited to a particular cell type. For example, an exemplary device in accordance with the disclosed subject matter can provide an environment for 3D cell culture with unconstrained cell migration (e.g., cell migration can occur within an extracellular matrix without constraint by pillars, posts, or other structures). In another embodiment, an exemplary device in accordance with the disclosed subject matter can provide tubular channels suspended in the extracellular matrix. That is, for example, the tubular channels can be completely lined with extracellular matrix and cells seeded in the channel need not be in contact with any physical surfaces (glass, PDMS, etc) of the substrate. In this manner, cells seeded into the channels can form lumens therein, as opposed to merely coating part of the interior surface of the channel. Cell migration into the extracellular matrix can be unimpeded by any physical surface, and therefore, unconstrained in any direction. For example, the direction of cell migration can be observed in response to a gradient of soluble factors established in the extracellular matrix.

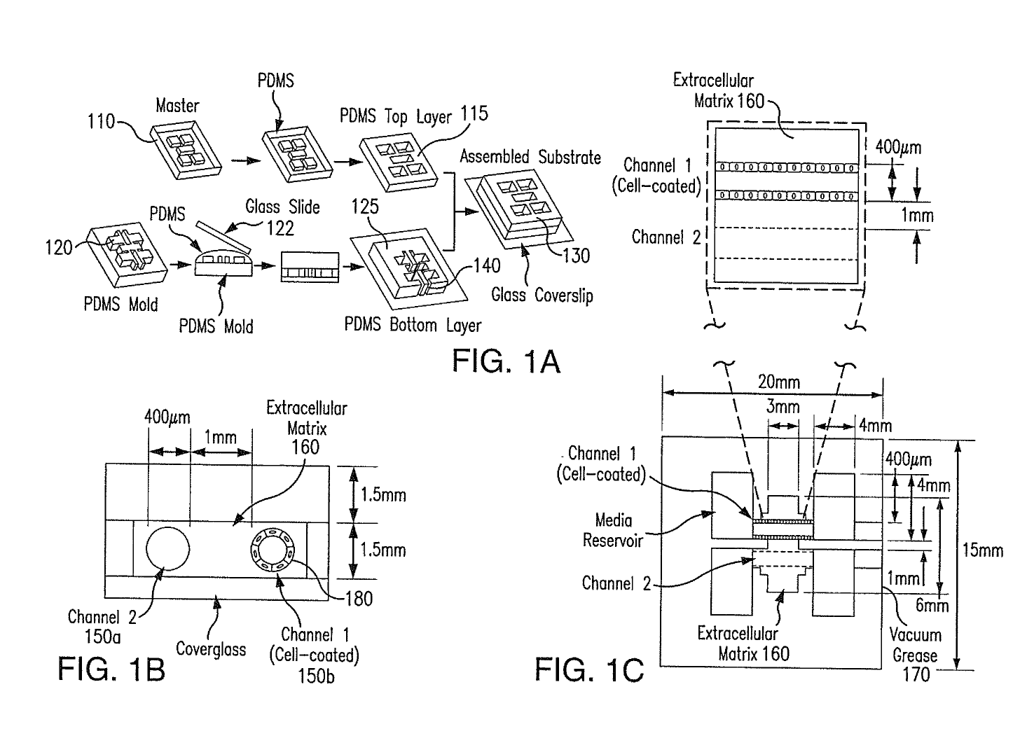

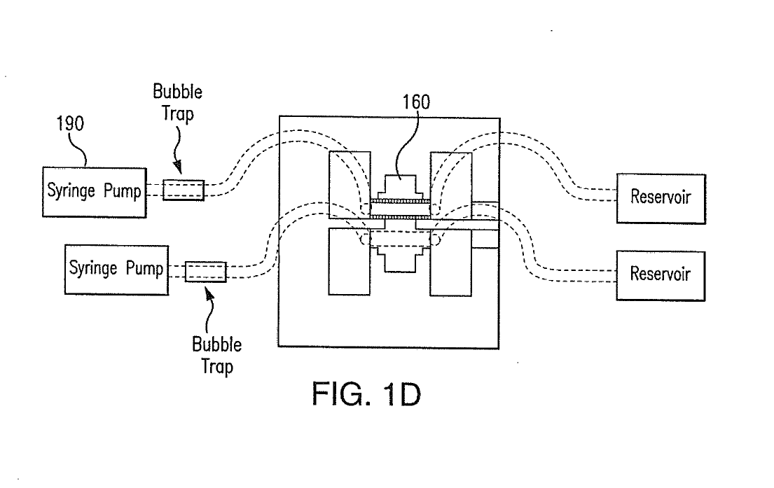

For purposes of illustration, and not limitation, FIG. 1A-D shows a schematic diagram of one embodiment of the presently disclosed subject matter. It should be noted that the figures depict a small subset of the potential geometries that could be configured. The channel number, arrangement, and geometry (e.g., diameter, length, regularity) can all be varied. As depicted in FIG. 1A-D, one embodiment includes two parallel channels of the same length and diameter, with each channel connected to two separate fluid reservoirs. Other variations (e.g., a single channel, a plurality of channels, different diameter channels, or the like) may be preferred for other applications. Additionally, the channel need not be open to or connected to a reservoir. For example, a channel could dead-end in the middle of the device, or have two closed ends. The channels can have, for example, a diameter of 200 μm. In alternate embodiments, the diameter can be larger or smaller, for example within the range of about 2-1000 μm. The space between the channels can be, for example, about 1 mm. However, in alternate embodiments the space between the channels can be larger or smaller. For example, the space between the channels can be in the range of about 0.1 mm to about 5 cm.

FIG. 1A depicts a schematic of a substrate assembled between two PDMS layers according to one exemplary embodiment of the presently disclosed subject matter. A top PDMS layer 115 can be cast, e.g., from a silicon or PDMS master 110. A bottom PDMS layer 125 can be peeled off from the PDMS mold 120 after being sandwiched between a glass slide 122 and PDMS mold 120 and cured (for example, at 65° C. in an oven). The two layers (115 and 125) can then be plasma treated and adhered together. The top layer can provide for taller reservoirs, thus increasing the volume and allowing for more media to be stored therein. Moreover, in certain embodiments, the height and dimensions of the reservoirs can be tuned to control fluid flow as described herein.

While FIG. 1A depicts assembly of the layers using a silicon or PDMS master, one of ordinary skill in the art will appreciate that various other techniques can be used. For example, and not limitation, the bottom layer can be 3D printed using biocompatible materials such that the reservoirs are tall enough to hold media and contain tubular compartments connected to the reservoirs and interstitial compartment. Alternatively, the bottom layer can be fabricated from customizable hole punchers and then adhered to a glass coverslip.

FIG. 1B-C depicts a top view and side view of the substrate. Channels 150 can be formed by inserting two acupuncture needles through microfabricated gaps 140 between the top and bottom PDMS layers before pouring a soluble extracellular matrix (ECM) 160. Needles can be coated with biocompatible, biodegradable, and sacrificial materials prior to insertion. The ECM 160 can be, for purposes of illustration and not limitation, fibrin, collagen, PEG, or other suitable ECM. Additionally, the ECM can also contain seeded cells. The ECM can encapsulate the needles. After gelation of the ECM is complete, the needles can be pulled out to create cylindrical channels 150.

It will be appreciated that in place of the acupuncture needles, other suitable techniques may be employed to create channels. For example, a sacrificial filament may be implanted in the ECM which can be subsequently dissolved. Alternatively, a suture or string can be placed in the ECM which can subsequently be removed to create a channel. 3D printing of biocompatible and biodegradable materials can also be employed to generate channels and network of channels. Non-limiting and exemplary methods to create channels in the ECM are known in the art, e.g., as disclosed in Chrobak K M, et al., Formation of perfused, functional microvascular tubes in vitro, Microvascular Research, 2006. 71(3):185-196 and Golden A P, et al., Fabrication of microfluidic hydrogels using molded gelatin as a sacrificial element, Lab on a Chip, 2007, 7:720-725.

After the needles are removed, vacuum grease 170 can be used to seal off the PDMS gaps 140. Endothelial cells 180 can be seeded into the channel to form a monolayer. The substrate can be placed on a rocker or ported with syringe pump 190 to enable flow through channels 150, as depicted in FIG. 1D. That is, for example, flow through tubular compartments can be gravity-driven, pressure-driven, or through cellular contraction (e.g., via cardiac cells generating contraction).

In one embodiment, for example, gravity-driven flow can be obtained by porting the ends of the channels with vertical fluid reservoirs where the fluid levels are at different heights and tunable. Fluid reservoirs can be connected to syringes to maintain differential fluid heights. Gravity-drive flow can additionally be generated by placing the substrate on a rocker. The rocker can be a piece of laboratory equipment comprising a platform that tilts back and forth on one axis or orbits around an axis. In some embodiments, the platform can tilt up to 30 degrees away from the horizontal plane in both directions. The substrate can be positioned on the rocker such that tilting causes the reservoirs connected to a given channel to be at different vertical planes. The fluids contained in the reservoirs are thus at different heights, resulting in a hydrostatic pressure difference which can drive flow through the channel. Thus, the rocker can cause fluid flow to occur in both directions in an oscillatory manner. A rocker used in conjunction with one embodiment of the presently disclosed subject matter, depicted in FIG. 1A-D, can achieve flow rate ranges from 0 μL/s to 4.4 μL/s. Flow rate can be adjusted with configuration changes such as channel diameter, length, and tilt. In another embodiment, the rocker can rotate around an axis to generate complex patterns of flows.

In other embodiments, a syringe pump 190 can be used, and can achieve arterial and supraphysiological flow rates. The reservoirs can be plugged with silicone tubing. The tubing can be attached to a syringe on a syringe pump 190 which pushes fluid through the tubing. In some embodiments, physiologic and supraphysiologic flow rates can be achieved. Mucus can flow at 2-5 mm/min, which can be equivalent to approximately 7 nL/s in the device configuration of one embodiment of the disclosed subject matter, disclosed in FIG. 1A-D, where two parallel channels of the same length and diameter are each connected to two fluid reservoirs. In this embodiment, arterial flow rates of about 9-27 μL/s can be achieved. Supraphysiological flow rates of approximately 90 μL/s can be achieved.

In other embodiments, a channel can be lined with contractile cells such as cardiac muscle cells or smooth muscle cells that can respond to stimulant(s) to generate contraction forces to constrict and dilate the channel thereby driving fluid flow through the channel.

In one exemplary embodiment, an exemplary device can be used to generate an organotypic model of angiogenesis. Two tubular compartments 150, approximately 400 μm in diameter and spaced approximately 1 mm apart, can be encased in collagen I gel. One of the tubular compartments 150b can be lined with endothelial cells and perfused with basal cell culture medium. The other tubular compartment 150a can be perfused with cell culture medium containing soluble angiogenic factors. The resultant diffusion of media into the interstitial collagen gel from both channels can create a stable angiogenic gradient, which stimulates matrix degradation and invasion, multicellular sprouting of endothelial cells, and formation of vessel lumens from the endothelialized cylindrical compartment in the direction of the source of the angiogenic factors.

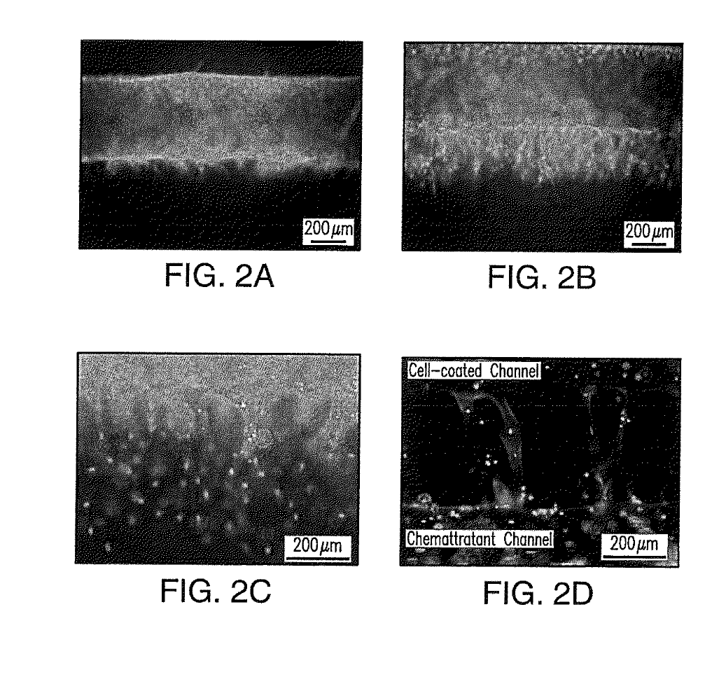

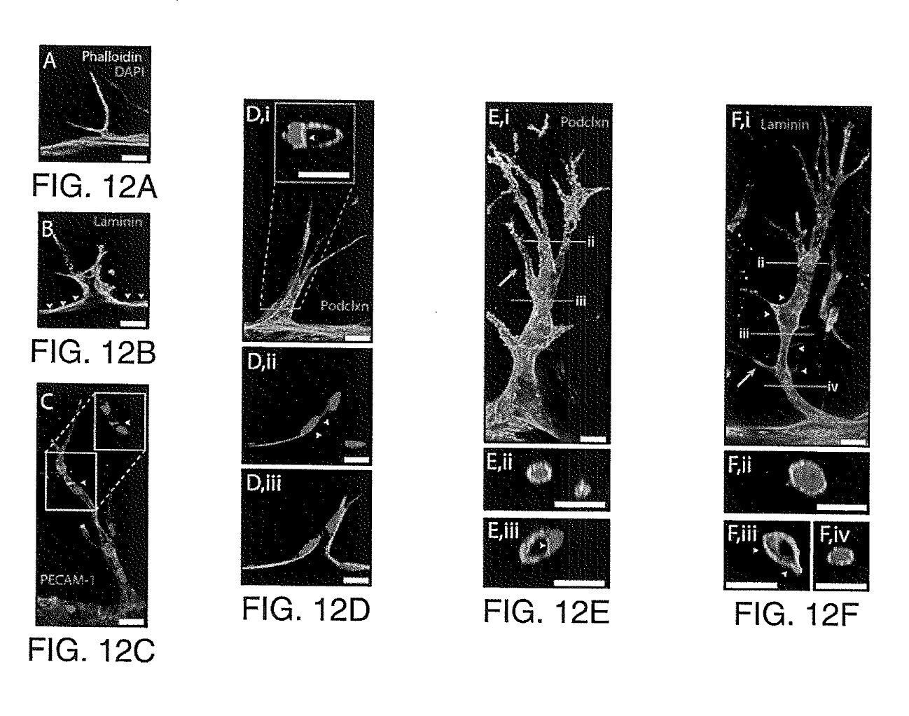

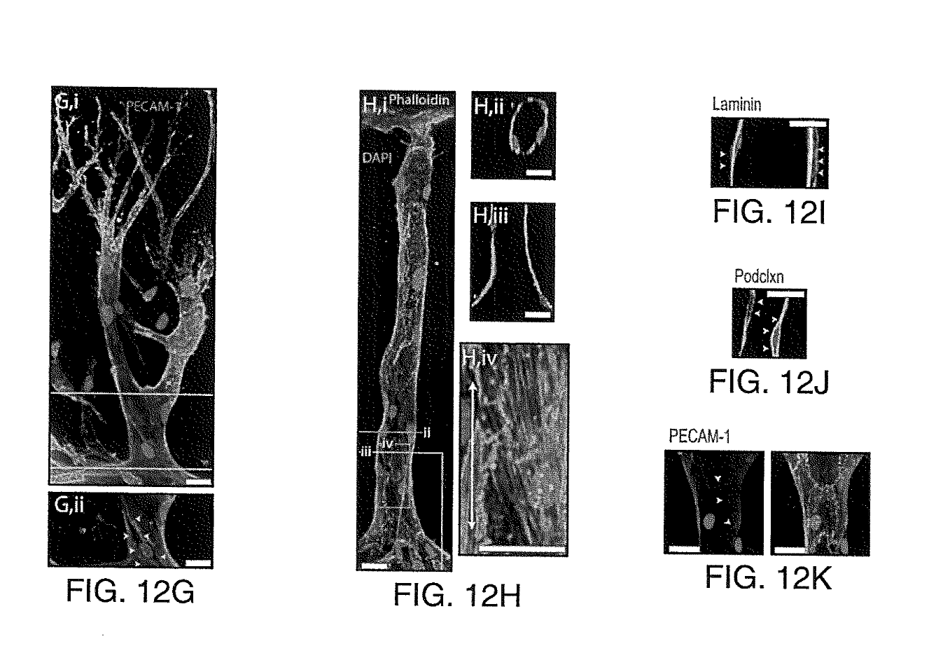

FIG. 2A-D is a depiction of endothelial cell sprouting and vessel development in a microfabricated 3D cell culture device according to one embodiment of the presently disclosed subject matter. FIG. 2A is an image of endothelial cells lining a tubular channel imitating invasion into the interstitial ECM after two days of exposure to angiogenic growth factors. The observed invasion is unidirectional (downwards) in the direction of an adjacent channel releasing the growth factors. FIG. 2B is an image of endothelial sprouts formed by day 4 of exposure. FIG. 2C is an image of lumen formation that occurred by day 7 of exposure. Fluorescent beads, e.g., 3 μm in size, can be perfused through the channel and enter the sprouts, and are shown in the image. FIG. 2D is an image of sprouts bridging two parallel channels which are fully perfusable by day 8. The fluorescent beads can be perfused through one channel and pass through the vessels to the other channel. Additionally, as described herein below in connection with an example of the disclosed subject matter, FIG. 12A-K illustrates the characterization of angiogenesis through multiple stages of the sprouting process from early sprouting (FIG. 12A,B,D), intermediate sprouting (FIG. 12C,E,F,G) until neovessel formation (FIG. 12H-K). Characterization shown in FIG. 12 describes localization of polarity proteins such as podocalyxin, deposition of basement membrane proteins such as laminin, formation of cell-cell junctions such as PECAM-1, lumen formation, cytoskeletal features such as filopodial protrusions stained by Phalloidin found in branching tip cells throughout the sprouting process (FIG. 12A-G) but regressed in neovessel (FIG. 12H).

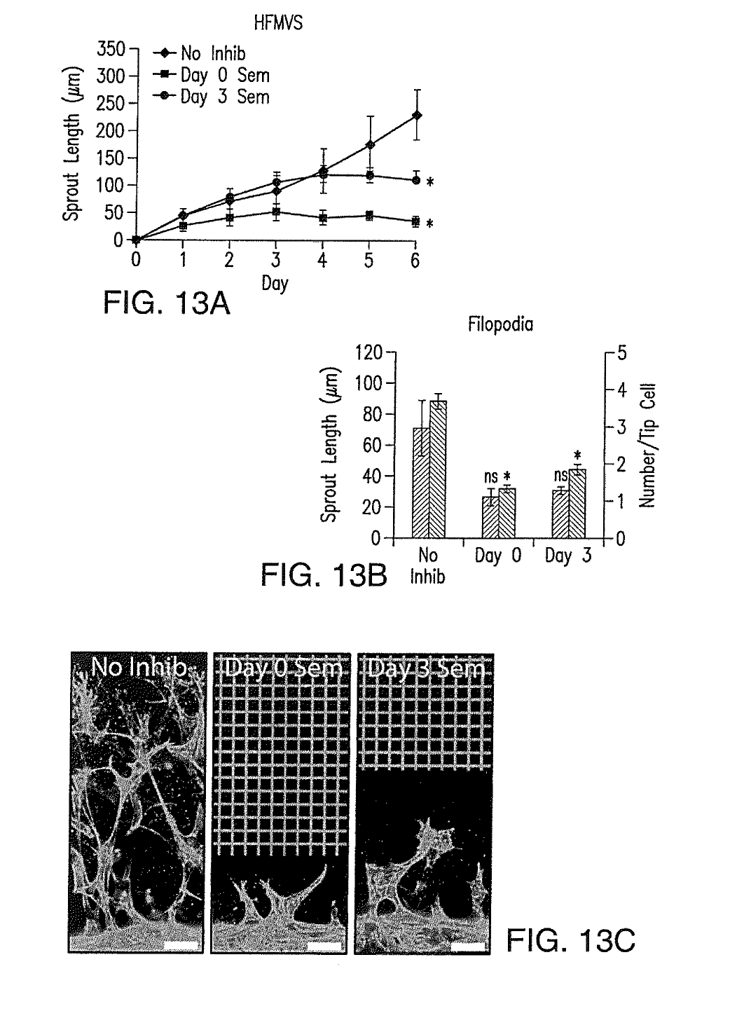

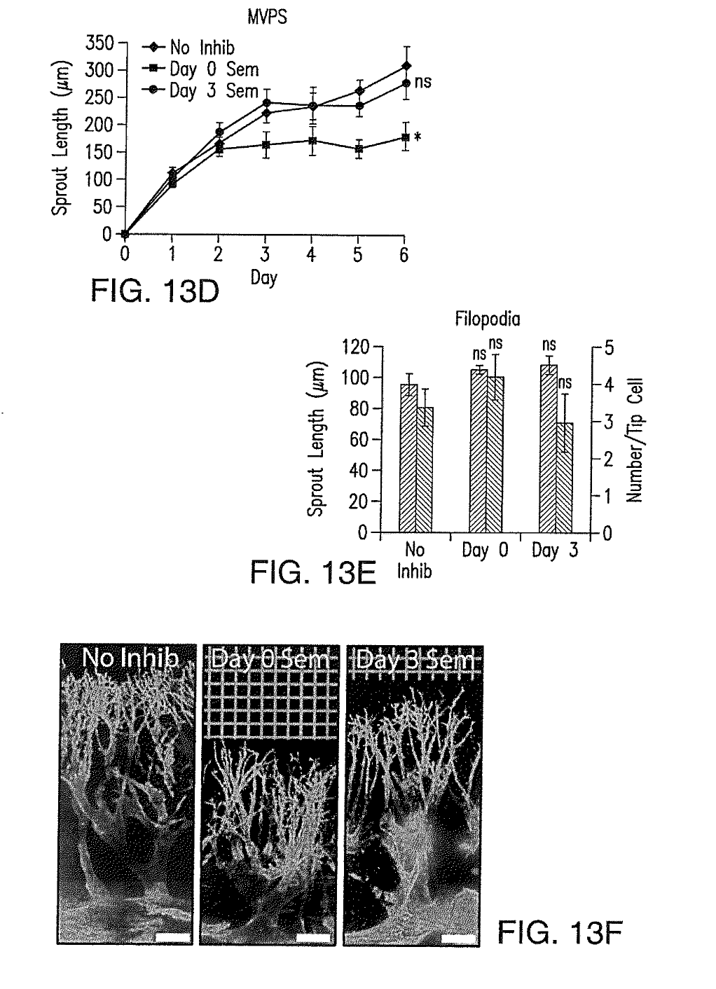

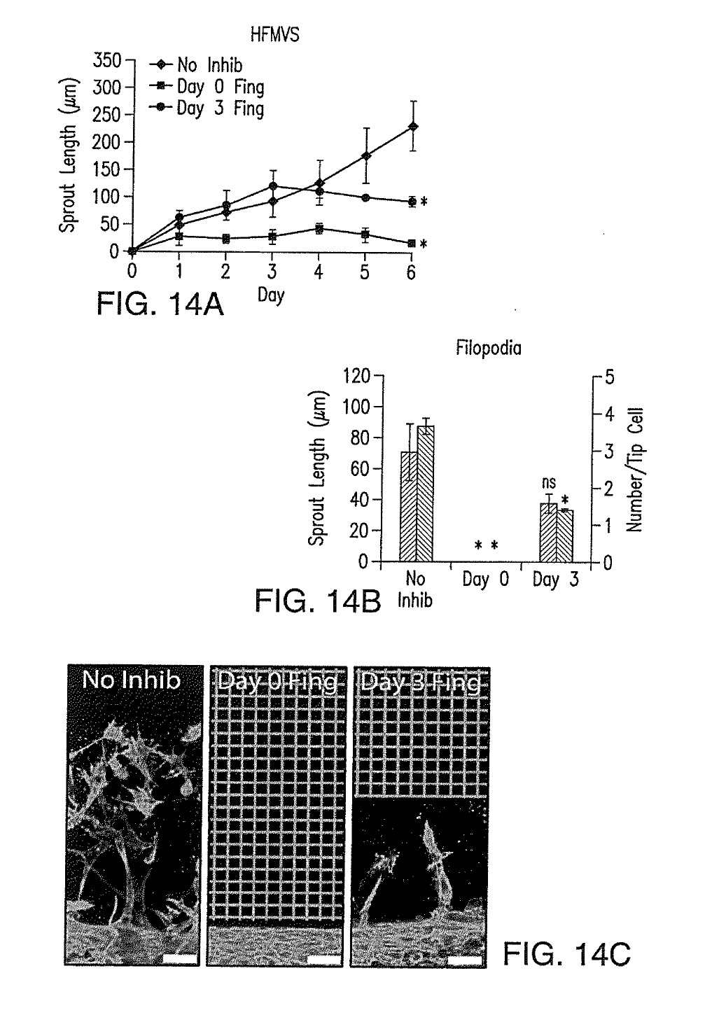

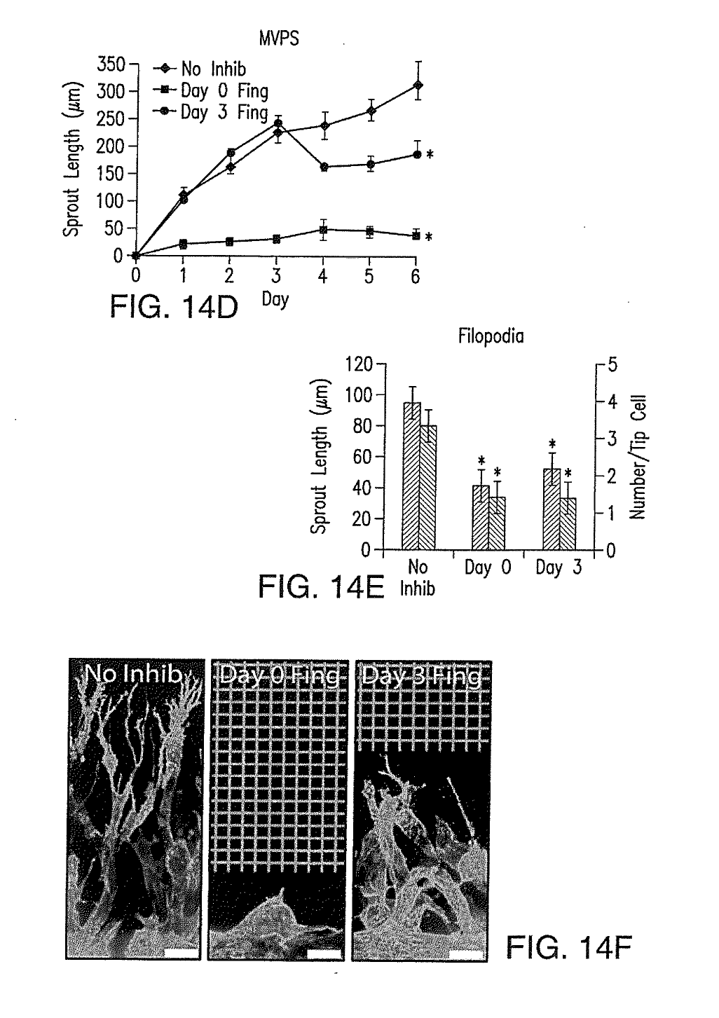

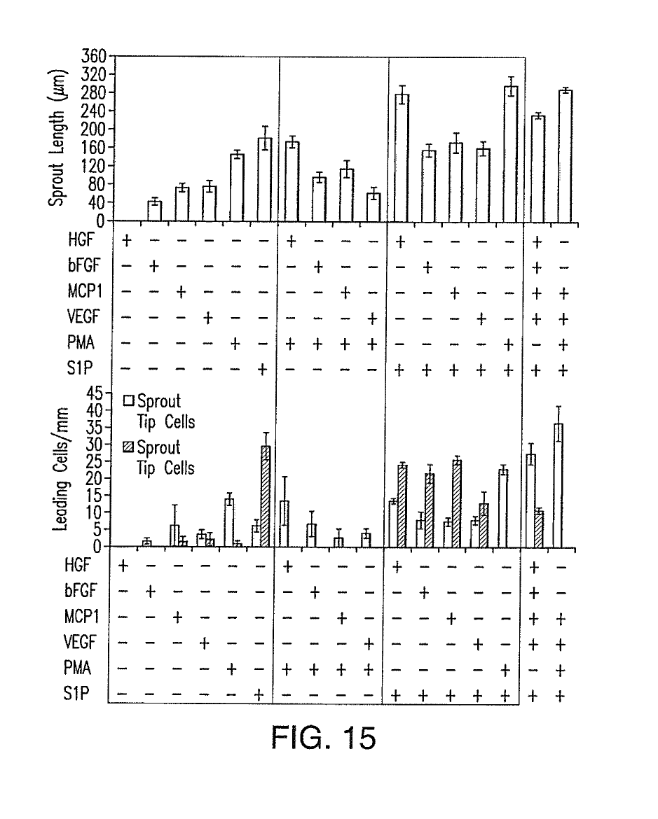

Furthermore, for purpose of example and not limitation, FIGS. 13-15 illustrate how an exemplary 3D cell culture device in accordance with the disclosed subject matter can be used to screen pharmacological drugs (FIGS. 13A-F and FIGS. 14A-F) and angiogenic factors (FIG. 15) that can influence the morphogenetic process of 3D angiogenic sprouting. FIG. 13 and FIG. 14 illustrate the effects and efficacy of Semaxanib (B) and Fingolimod (C) to inhibit angiogenic sprouting driven by two different angiogenic cocktails (MVPS, and HFMVS) in accordance with observations from an example of the disclosed subject matter described herein below. FIG. 15 illustrates the effectiveness of a single angiogenic factor or combination of factors to trigger sprouting in accordance with observations from an example of the disclosed subject matter described herein below. Pro-angiogenic screening can identify a combination of angiogenic factors that can induce robust sprouting such as combinations of MCP-1, VEGF, PMA, S1P (MVPS) or HGF, bFGF, MCP-1, VEGF, S1P (HFMVS).

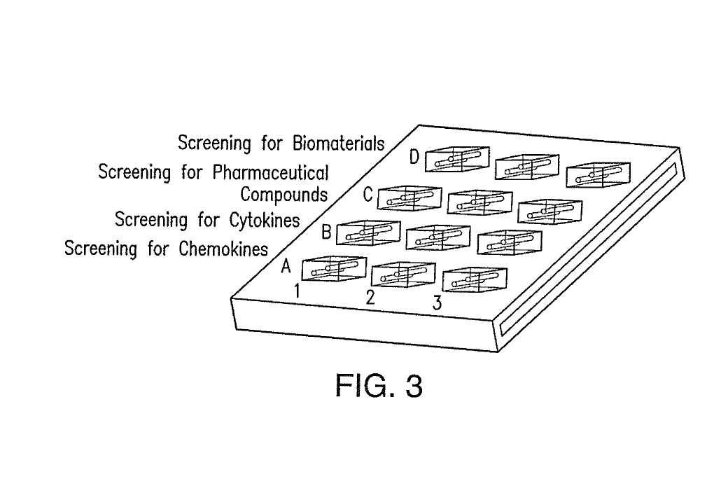

In another embodiment, the device disclosed above can be scaled up into a multi-well format, e.g., for use in high-throughput assays. This arrangement can be used, for example, to screen the effects of specific growth factors, cytokines, small molecules, ECMs, and non-endothelial cells (e.g., fibroblasts, immune cells) on angiogenesis or other cellular processes. As depicted in FIG. 3, each well can contain an individual compartmentalized 3D cell culture system to allow, for example, screening of chemokines, cytokines, pharmaceutical compounds, and/or biomaterials simultaneously to determine their angiogenic potentials. It should be noted that although FIG. 3 depicts twelve 3D culture systems, a larger format may also be fabricated to accommodate multi-parameter screening. Additionally, this embodiment may be used as a test bed to evaluate the efficacy of candidate anti-angiogenic compounds before administering these compounds in pre-clinical studies.

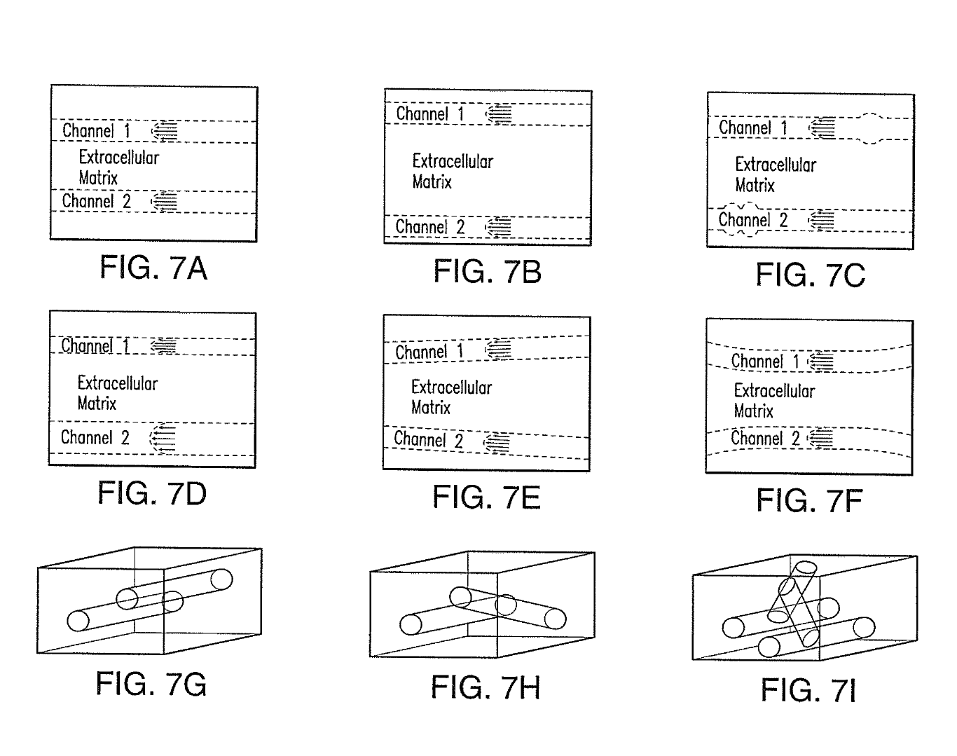

One of ordinary skill in the art will recognize that a variety of other configurations are contemplated within the spirit and scope of the presently disclosed subject matter. For example, and with reference to FIG. 7A-I, in the simple case of two tubular compartments and one interstitial ECM compartment, the device can be designed in different configurations, such as spacing the channels farther apart or closer together, and/or changing the diameters of one or both channels. Additionally, the channels may be molded such that they are not parallel or co-planar. In yet other embodiments, more than 2 channels may be fabricated. Channels can be oriented as true networks that intersect with the interstitial ECM compartment. Additionally channels may also share fluidic reservoirs. Such variants can enable complex gradients and laminar/turbulent flow profiles when used in conjunction with perfusion, and can be useful for examining how spatial heterogeneities in the microenvironment affect cellular processes such as angiogenic sprouting and cell extravasation from the channels.

FIG. 7A-I depicts a number of different spatial configurations which can be embodiments of the presently disclosed subject matter. FIG. 7A-F reveal top-view illustrations of the tubular compartments. Spatial parameters such as diameter, shape (e.g., rectangular or circular cross section), distance between the compartment, and roughness of the tubular components (e.g., curvature and nooks), can be changed. FIG. 7G-I reveal 3D spatial organization of the tubular compartments with respect to one another. The tubular compartments may be aligned in planar, non-planar, and skewed orientations as illustrated in FIG. 7G-H. Complex tubular networks can also be arranged including intersecting tubular compartments, as illustrated in FIG. 7I. Additionally, a separate fluid compartment can be included above or below the ECM and/or channels, or some of the channels. It will be appreciated that the configurations just disclosed can be applied in combination with various aspects of the presently disclosed subject matter and other embodiments disclosed herein.

In another aspect of the presently disclosed subject matter, the device can be used in a method for culturing cells. In some embodiments, the device can be used to identify the mechanisms of action of test compounds and materials. Well known genetic tools such as RNA interference and genetic knockout can be applied to cells cultured in the device to identify gene targets for modulating angiogenesis or other cellular processes.

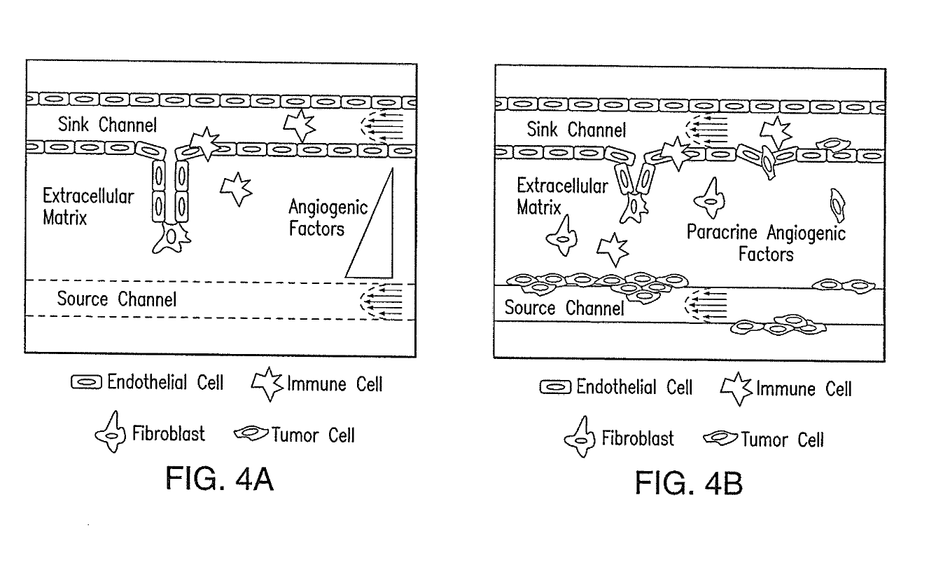

In other embodiments, the device can also be used as a diagnostic test for personalized medicine. For example, cancer cells extracted in biopsies can be cultured within a tubular or interstitial compartment to determine whether they attract angiogenic sprouting from an adjacent endothelialized tube. Moreover, and with reference to FIG. 4A-B, anti-angiogenesis drugs can be administered in this assay to determine whether patient-specific cancer cells are responsive to anti-angiogenesis treatment. The device can also be configured to test compounds that do not directly modulate angiogenesis. Certain cancers can overexpress cell surface receptors that bind nonangiogenic growth factors such epidermal growth factor (EGF). The responsiveness of these cells to anti-cancer therapeutics can be studied in the absence of endothelial cells. For example, patient derived breast cancer cells can be cultured in an interstitial ECM compartment formed with Matrigel. In this environment, these cells can form irregular spheroidal masses known as mammary acini. Growth factors such as EGF or pharmacological compounds that target EGF can be perfused through the tubular compartments. In this scenario, inhibition of tumor cell migration and proliferation can be the metrics of drug efficacy instead of angiogenic sprouting. In these different configurations, the device can provide useful information as to whether a patient will respond favorably to a particular treatment.

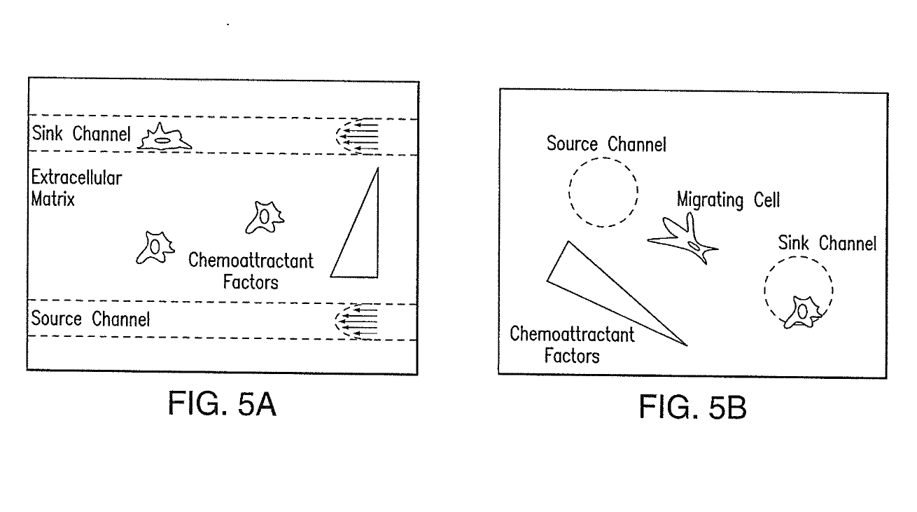

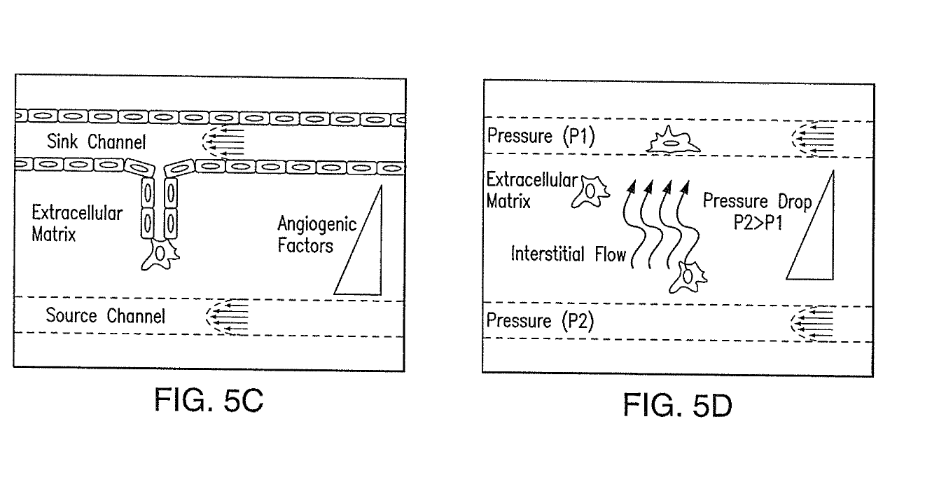

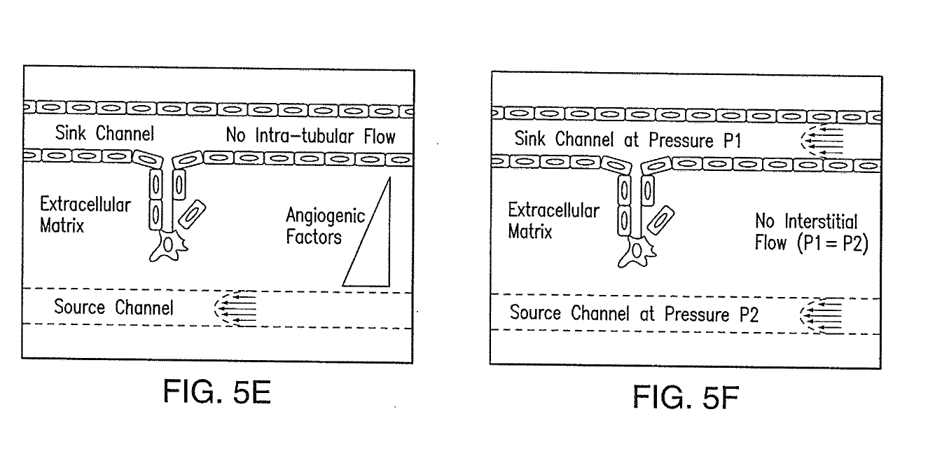

In other embodiments, the device can be used to study any cellular process guided by gradients of soluble factors. For example, with reference to FIG. 5A-F, the tubular compartment can be perfused with fluids containing any type and concentration of soluble factors to generate stable gradients of defined composition and steepness. For example, and with reference to FIG. 5A-B, the device can be used to study single cell 3D migration in the presence of a chemokine gradient. Immune cells, endothelial cells, epithelial cells, neuron cells and other cells can be cultured in one of the tubular compartments or in the interstitial ECM compartment and subjected to gradients of chemokines. FIG. 5A depicts an exemplary illustration of single cell 3D migration in the presence of gradients of chemokines, cytokines, metabolites, toxins, or pharmacological compounds in accordance with the disclosed subject matter. FIG. 5B depicts an exemplary illustration of cell migration in all directional axes which can be observed by positioning tubular components in different planes. This can be shown by the device cross section in which the source channel is positioned higher than the sink channel in the extracellular matrix.

In another example, with reference to FIG. 5C, multicellular processes, such as lymphangiogenesis, neuronal sprouting, mammary gland development and branching morphogenesis can be observed in 3D in the presence of chemoattractant gradients. Gradients are not limited to chemokines—other types of molecules can be spatially distributed in gradients including oxygen, toxins, metabolites (e.g., nitric oxide, lactic acid, glucose), and pharmacological compounds (including inhibitors and activators). Tubulogenesis of neuronal cells and epithelial cells from different tissues, including pancreas, lung, trachea, kidney, and mammary gland can be used.

In yet another example, with reference to FIG. 5D-F, in addition to fluid composition, rate of perfusion, direction of flow, and flow patterns (for example, laminar, turbulent, pulsatile, or multiphase) can be controlled separately in the two tubular compartments to modulate pressure drops, shear forces and interstitial flow within the tubular and interstitial ECM compartments. As such, another use of the device is to examine how fluid mechanics parameters affect single cell and multicellular processes in 3D. In one specific example, the effect of fluid perfusion on the stabilization of nascent blood vessels can be determined. Here, angiogenic gradients can be used to stimulate angiogenic blood vessel formation. Subsequently, the gradients are removed while cell culture media is continually perfused to maintain a pressure differential and interstitial flow across the ECM compartment. FIG. 5D depicts an exemplary illustration of the modulating of perfusion direction (co-current, counter-current), rate, and pressures according to embodiments of the disclosed subject matter to generate different flow patterns within the tubular and interstitial compartments. Altering flow parameters can affect single and collective cell migration. FIG. 5E depicts an exemplary illustration of the effect of intra-vascular flow on vessel stabilization. FIG. 5F depicts an exemplary illustration of the effect of interstitial flow on vessel stabilization.

In yet other embodiments, the device is used to investigate the effect of heterotypic cell interactions on cell function. For example, with reference to FIG. 4A-B, numerous physiologic and pathophysiologic processes such as angiogenesis, lymphangiogenesis, neuronal sprouting, and cancer metastasis can involve the activities of more than one cell type. In angiogenesis, for example, fibroblasts, tumor cells and macrophages can all secrete paracrine factors that can direct endothelial sprouting. Moreover, nascent blood vessels subsequently can be stabilized by pericytes and smooth muscle cells. These types of heterotypic cell interactions can be studied in the disclosed device by co-culturing different cell types within the same compartments or in different compartments.

In one embodiment, as depicted in FIG. 4A, endothelial cells can be cultured first within a tube followed by the addition of immune cells and inflammatory cytokines to model the inflammatory response. As depicted in FIG. 4A, endothelial cells in one tubular compartment can sprout towards a gradient of angiogenic factors extending across the interstitial compartment. Immune cells can be distributed either within the tubular compartments or in the interstitial compartment to study the effects of immune cells on vessel development. Moreover, extravasation of immune cells (e.g. neutrophils, monocytes and macrophages) between the channel and ECM can also be studied.

In another embodiment, as depicted in FIG. 4B, endothelial cells can be cultured in one channel while tumor cells can be cultured in the second channel to model tumor-induced angiogenesis and metastasis. Here, the tumor cells can release paracrine factors that stimulate endothelial sprouting and invasion into the ECM. Rather than culture cells only in the tubular compartments, cells can be introduced into the interstitial ECM compartments as well. For example, fibroblasts or mesenchymal stem cells can be dispersed within the ECM while one or more of the tubes are lined with endothelial cells. This can be one approach to study the effects of stromal cells on vascularization.

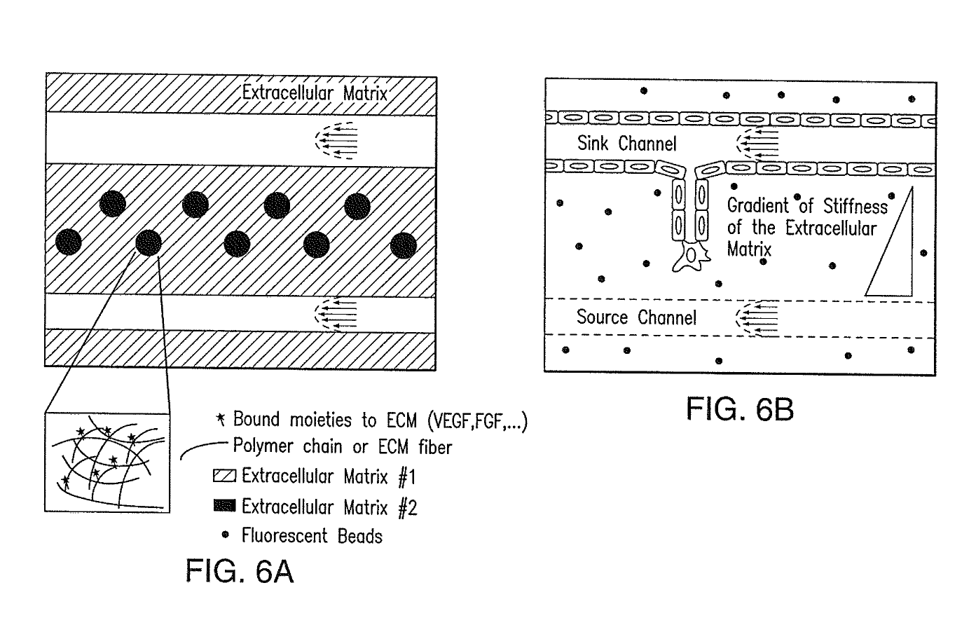

In yet other embodiments, with reference to FIG. 6A-B, the device can be used to study how extracellular matrix properties, such as stiffness, porosity, and ligand composition affect cell function. The tubular structures can be encased in different native or synthetic ECMs that present a wide range of mechanical, adhesive and functional properties.

In one embodiment, with reference to FIG. 6A, the interstitial compartment can be comprised of fibrin or polyethylene glycol hydrogels that have been cross-linked with angiogenic growth factor-binding peptides. These materials can potentially alter chemoattractant gradients by capturing soluble factors, and can therefore modulate angiogenic sprouting from the endothelialized tubular compartments. In another embodiment, the interstitial compartment can be comprised of collagen matrix of varying density, porosity and degree of cross-linking. For example, in FIG. 6A, a patterned matrix (illustrated as circles) can be coupled with chemical and biochemical moieties that are MMP-sensitive to enable a slow release profile of chemokines, cytokines, and pharmaceutical reagents. Endothelial cells or tumor cells can be cultured in the tubular compartments and subjected to soluble gradients as done in previous configurations. Therefore, this system can be used to understand the role of extracellular matrix mechanics in the regulation of tumorigenesis and angiogenesis.

As depicted in FIG. 6B, fluorescent beads (illustrated as small dots) can also be embedded within the interstitial ECM as fiduciary markers for visualizing matrix deformations and traction forces caused by cellular movements. As another example, the ECM within the interstitial compartment can be spatially patterned, as depicted in FIG. 6A. One type of ECM, such as fibrin can be dispensed within a void in the compartment and surrounded by collagen I. Alternatively, the interstitial compartment can be filled with a photopolymerizable hydrogel that has been non-uniformly cross-linked to create regions of varying stiffness. In these embodiments, durotactic or haptotactic migration of cells from the tubular compartments into the interstitial compartments can be studied.

The disclosed subject matter provides for the mimicking of in vivo microenvironment for a plurality of types of cell-based assays, including, but not limited to: angiogenesis, inflammation, tumorigenesis, tubulogenesis, proliferation, migration, differentiation, and signaling assays. The disclosed subject matter also provides for the evaluation of cellular migration, endothelial cell sprout migrations, anastomosis, vessel lumen formation, immune cell trafficking, and tumor mass migration. The disclosed subject matter has broad applicability in drug discovery, tissue engineering and basic cell biology.

In addition to endothelial sprout migrations, anastomosis and vessel lumen formation, the disclosed subject matter can also have application in immune cell trafficking (i.e., extravasation) and tumor cell migration (as seen in metastasis). In these embodiments, endothelium can be absent. For example, in one embodiment, only immune cells (for example, neutrophils) can be cultured in the channels while endothelial cells can be omitted. The movement of immune cells between the tubular and interstitial compartments can be observed.

Moreover, the presently disclosed subject matter has can be applied to models of different types of vasculature—lymphatic vessels, veins, arteries, and capillaries all contain endothelial cells but these endothelial cells can express different markers. For example, endothelial cells from microvasculature (capillaries) can respond differently to angiogenic factors than venous endothelial cells. Thus, tissue-specific endothelial cells can be used in the substrate to generate more physiologically relevant models of blood vessels and lymphatic vessels. Furthermore, other cell types, notably smooth muscle cells, can form part of the arterial vasculature. For example, a thick smooth muscle wall layer surrounding the endothelium. The substrate disclosed herein can incorporate this additional complexity.

In some embodiments, smooth muscle cells can be cultured inside the tubular compartment, followed by the addition of endothelial cells to create a multi-layer vessel analog. In another embodiment, brain cells may be seeded initially, followed by endothelial cells serving to model the blood-brain barrier. Additionally, the disclosed subject matter can be applied to models of non-vascular tissues disclosed herein. Other glandular tissues such as pancreas (epithelialized tubes which are filled with digestive enzymes), breast (lactiferous ducts that carry milk), liver (bile ducts that carry bile), brain (ventricles that contain cerebrospinal fluid), intestines (that transports food) and kidney (renal tubules that carry waste filtrate) can be modeled. For example, in one embodiment, kidney epithelial cells can be seeded into the tubular compartment to form an artificial renal tubule. Solutions containing metabolites can be flowed into the tubular compartment to assess the re-absorption function of the artificial renal tubule. The artificial renal tubule can be treated with pharmacological toxins which can impair the ability of the cells to re-absorb metabolites from the filtrate solutions.

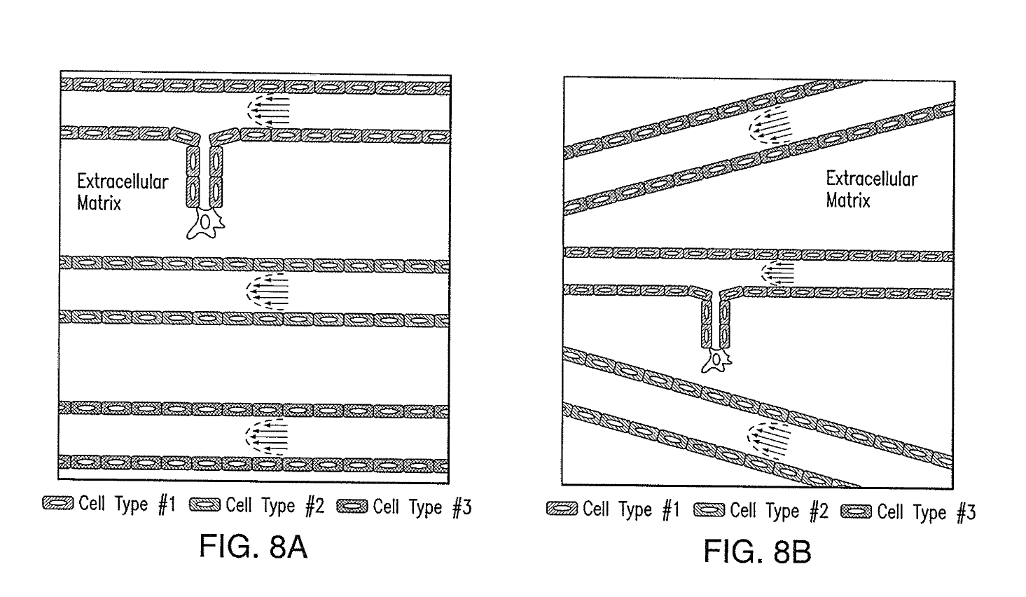

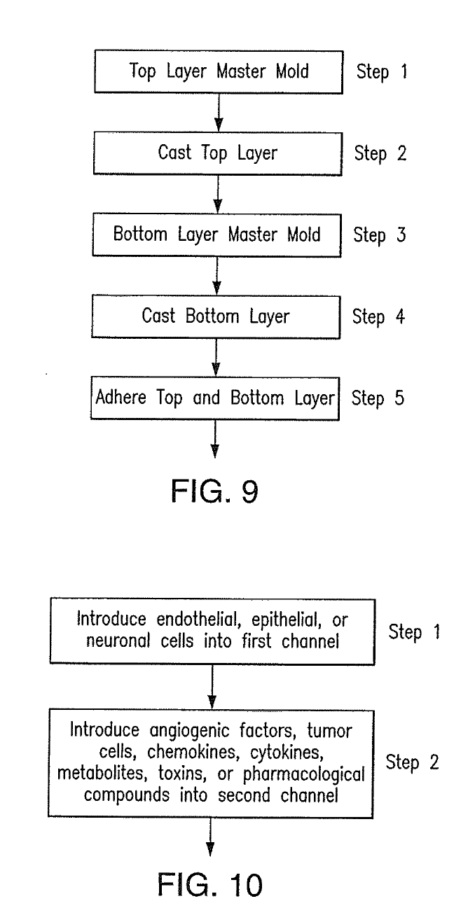

With reference to FIG. 8A-B, a network of multiple channels can be used to mimic tissue with coexisting tubular networks such as, for example, brain, vasculature, pancreas, liver, gall bladder, spleen, intestine, mouth, nasopharynx, esophagus, peritoneal cavity, lung, trachea, kidney, bladder, ureter, prostate, and mammary gland tissue. For example, some channels can be lined with vascular endothelial cells while some channels can be lined with lymphatic endothelial cells to study the cellular communication and interaction including but not limited to nutrient and metabolites transports between these two networks. In another example, a network of artery branches, vein branches, and bile ducts can be generated by lining different channels with different cell types to mimic and study cellular interactions and lobule circulation in liver. Spatial arrangement of the channels can be varied to study the effects of geometry and spatial arrangement in development of coexisting tubular networks, cellular interactions and transport in these coexisting tubular tissues.

### Examples

The present application is further described by means of the examples, presented below. The use of such examples is illustrative only and in no way limits the scope and meaning of the invention or of any exemplified term. Likewise, this application is not limited to any particular preferred embodiments described herein. Indeed, many modifications and variations of the invention will be apparent to those skilled in the art upon reading this specification. The invention is to be understood by the terms of the appended claims along with the full scope of equivalents to which the claims are entitled.

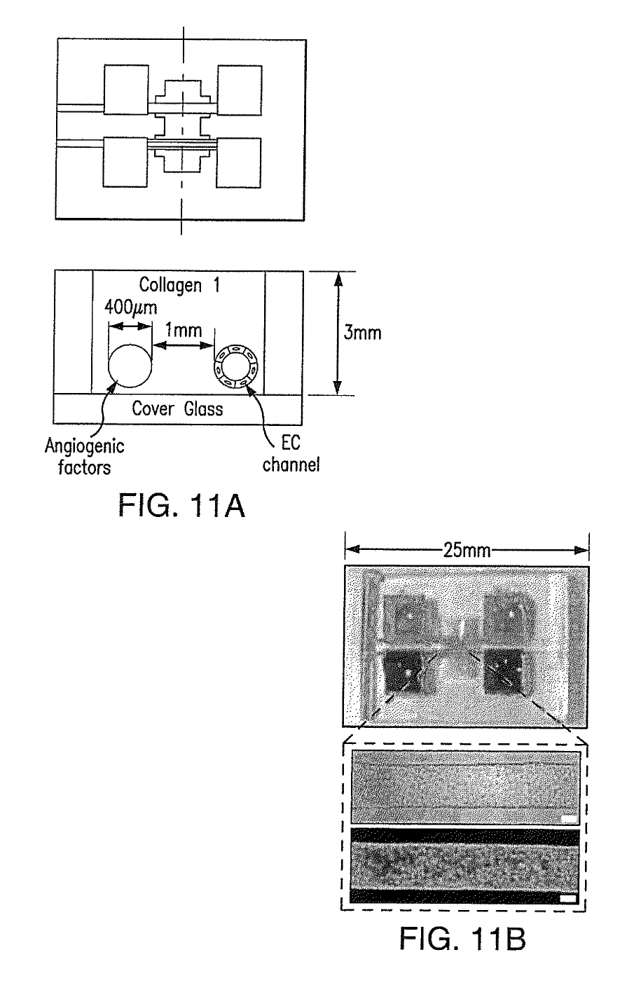

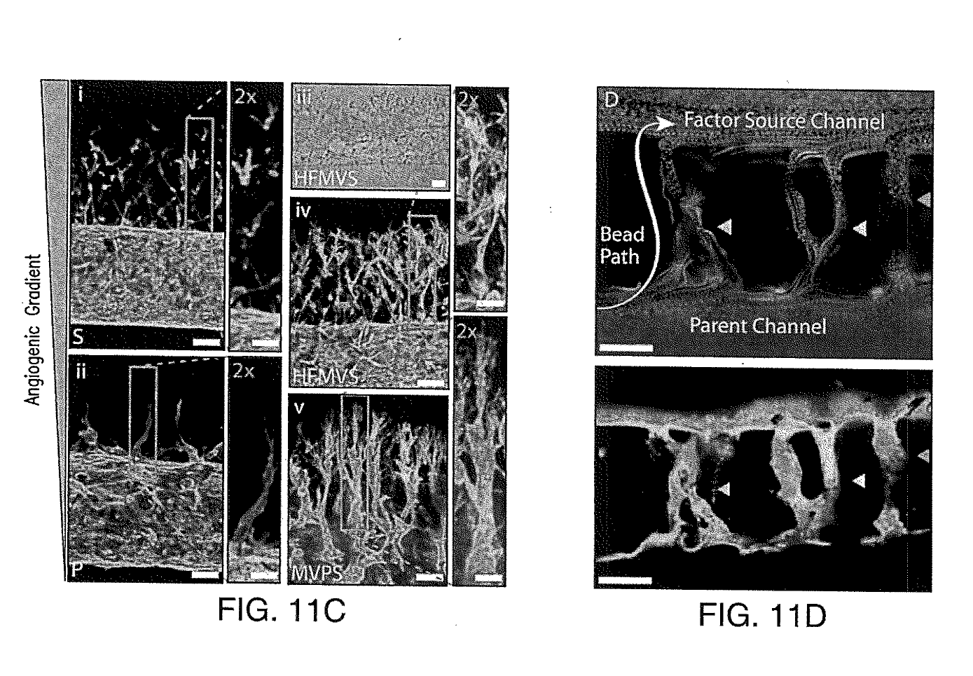

Examination the processes of angiogenic invasion and sprouting from an existing vessel in accordance with the disclosed subject matter will be described, with reference to FIG. 11A-D through FIG. 14A-F, for purpose of illustration and not limitation. Generally, a device in accordance with an embodiment of the disclosed subject matter can be prepared with an endothelium lining a cylindrical channel, surrounded by matrix, and exposed to a gradient of angiogenic factors emanating from a parallel source channel 150a. The device can be assembled by casting Type I collagen into a PDMS mold/gasket with two parallel needles held across the casting chamber. Upon collagen polymerization, the needles can be extracted to create hollow cylindrical channels in the collagen matrix 160. Endothelial cells (ECs) can be injected into one of the channels, allowing them to attach on the interior wall and form a confluent endothelium or “parent vessel” 150a. Flow can be maintained through both channels and media containing angiogenic factors can subsequently be added to the second channel to establish a gradient across the collagen matrix to the endothelium, as illustrated in FIG. 11B.

### Example #1

In a first example, the impact of various pro-angiogenic factors on directed invasion and sprouting from the parent vessel is examined. Six common factors associated with angiogenesis in the literature were selected: basic fibroblast growth factor (bFGF), hepatocyte growth factor (HGF), vascular endothelial growth factor (VEGF), monocyte chemotactic protein-1 (MCP-1), sphingosine-1-phosphate (S1P), and phorbol 12-myristate 13-acetate (PMA). After these factors were added individually to the non-endothelialized source channel, phase-contrast and confocal microscopy were used to assess the organization and development of EC invasion over four days. In connection with this example, for purpose of illustration and not limitation, VEGF, MCP-1, HGF or bFGF alone did not induce significant invasion (e.g., single cell invasion or collective cell invasion quantified by length of invasion and/or density of invasion) into the matrix, while S1P and PMA resulted in substantial directed invasion. This invasion was oriented directly toward the source channel, despite the fact that cell migration from the endothelium was not artificially constrained in any direction by the device used in connection with this example (FIG. 11C).

In connection with this example, for purpose of illustration and not limitation, S1P and PMA stimulated different modes of cell migration. S1P drove chemotactic migration primarily of single cells from the endothelialized channel, whereas PMA triggered collective cell migration that manifested itself in the form of sparse, long, multi-cellular sprouts into the matrix (FIG. 11Ci,ii). Progressively more complex combinations of the six factors yielded more substantial multicellular sprout-like structures, especially in the case of two distinct combinations that drove robust sprouting—HGF, bFGF, MCP-1, VEGF, and S1P (HFMVS) and MCP-1, VEGF, PMA, and S1P (MVPS). HFMVS-guided invasion exhibited numerous sprout-like structures that extended hundreds of micrometers from the endothelialized parent vessel as well as large numbers of solitary cells migrating into the matrix (FIG. 11Ciii,iv). The MVPS cocktail induced an even greater multicellular sprouting response with less single cell migration (FIG. 11Cv). In both cases, the sprouts continued to invade toward the source channel as long as the gradient was maintained.

When the tips of these sprouts reached the source channel (typically after one week), they breached into the source channel, forming new microvessels connecting the two parallel channels (FIG. 11D). To test whether these “neovessels” possessed functional, perfusable lumens, 3 μm fluorescent beads were added to the media flowing into the endothelialized parent channel. Beads traveled through the neovessels to the source channel with no leakage into the interstitial space, indicating fully developed lumens lined by a continuous endothelium. Overlaying frames of the time-lapse images demonstrate the path of the beads through these occasionally branching neovessels (FIG. 11D).

### Example #2

In a second example, changes in cellular organization during early stages of invasion were examined, in connection with the MVPS cocktail. Prior to stimulation, cells in the endothelialized channel exhibited the expected apical-basal polarity as demonstrated by the localization of CD34 apical marker podocalyxin to the luminal face. On the basolateral side of the endothelium laminin deposition was observed. Upon stimulation, occasional single ECs began invading into the matrix and extending filopodia-like protrusions in the direction of the angiogenic gradient (FIG. 12A). During initial invasion, interruptions in laminin immunofluorescence were observed, consistent with focal degradation of the basement membrane (FIG. 12B). These leading tip cells were replete with filopodia-like protrusions, morphologically recapitulating in vivo sprout tips. As these tip cells migrated deeper into the matrix, neighboring cells followed while maintaining cell-cell contacts along the length of the sprout, as shown by PECAM-1 staining (FIG. 12C). Thus, the sprouting process from the parent endothelium into the matrix involved collective cell migration that supported a contiguous structure between the sprout and parent vessel. Even at this early stage of 2-3 cells per sprout, evidence of lumen formation was detected in 3D reconstructions of confocal images (FIG. 12D). Moreover, apical-basal polarity appeared intact in the sprouts as evidenced by apically targeted podocalyxin staining (FIG. 12Di,iii).

As the sprouts continued to invade and extend into the matrix, they became longer, contained progressively more cells, and began to branch (FIG. 12E-G). Stereotypical sprouting morphology was evident in these mature sprouts, with cells at the sprout tip developing numerous thin filopodia-like protrusions, in contrast to cells in the stalk containing few filopodia protrusions (FIG. 12E-G). Lumens developed in both early sprouts (e.g., containing less than three cells per sprout) and late sprouts (e.g., having greater than or equal to three cells per sprout) that often extended from the parent vessel up to, but never within, the tip cell (FIG. 12D,E). Partial lumens occasionally were evident behind the tip cell that were not connected to the parent vessel, suggestive of spontaneous, focal cord-hollowing or lumenization (FIG. 12Fiv). Staining confirmed that the sprout tip cells lacked specific localization of podocalyxin, while stalk cells demonstrated localization of podocalyxin to the luminal space (FIG. 12E). Laminin deposition in the mature sprouts was observed (FIG. 12F) and PECAM-1-positive cell-cell junctions were generally intact throughout the sprouts (FIG. 12G). In addition to primary sprouts, maturation of secondary branches were also observed. Different stages of secondary branching were evidenced by stalk cells occasionally marked by direct filopodia-like protrusions suggesting early branch initiation (FIG. 2F), whole cells extending out from the stalk of the sprout (FIG. 2E), and finally as full multicellular branches with their own new tip cells extending toward the angiogenic gradient (FIG. 12G).

Upon formation of neovessels spanning the two channels, non-perfused filopodial protrusions disappeared (FIG. 12Hi). The neovessels were lumenized end-to-end (FIG. 12Hii, iii), and cells were aligned with flow as in the parent vessel, demonstrated by actin stress fiber alignment (FIG. 12Hiv). Additionally, deposition of laminin around the neovessels (FIG. 12I), localization of podocalyxin to the luminal domains (FIG. 12J), and PECAM-1 staining reflective of intact cell-cell junctions (FIG. 12K) were observed.

### Example #3

In a third example, the physiological response of sprouts to agents known to perturb the angiogenic process was examined. First, a VEGF receptor 2 (VEGFR2) inhibitor Semaxanib was added with the HFMVS angiogenic cocktail. If added from the outset, the inhibitor abrogated sprout initiation (FIG. 13A). Because angiogenic inhibitors are also thought to lead to regression of pre-existing sprouts, the effects of adding Semaxanib to the source channel after 3 days of uninhibited sprouting was examined. Further progression of sprouts was arrested, but obvious regression of the sprouts did not occur (FIG. 13A). Inspection of VEGFR2-inhibited sprout architectures revealed a near complete loss of the many filopodia-like protrusions normally present in the tip cells, with a decrease in the number and length of protrusions (FIG. 13B,C). Sprouting induced by the MVPS cocktail, while slowed, appeared to proceed despite VEGFR2 inhibition (FIG. 13D). Confocal images revealed that the filopodia-like protrusions in these sprouts were largely unaffected by Semaxanib, whether added at Day 0 or Day 3 (FIG. 13F). Quantitative analysis, as described herein below, showed that the number of filopodial extensions was unchanged and their length was unaffected (FIG. 13E).

### Example #4

In a fourth example, the morphogenetic responses to anti-angiogenic factors was examined with reference to the effects of perturbing S1P signaling, which acts as a strong chemoattractant through a G-protein coupled receptor (S1PR) and is known to regulate angiogenesis. Exposing cells to the S1PR inhibitor Fingolimod resulted in abrogation of sprout initiation when introduced at Day 0, and inhibited further sprout extension when given at Day 3 (FIG. 14A-F). These effects were observed as independent of which angiogenic cocktail (HFMVS or MVPS) was employed (FIG. 14A,D). Quantification of the remaining sprout structures revealed nearly complete loss of filopodia-like protrusions, with cells appearing less elongated and organized (FIG. 14B,C,E,F).

### Materials and Methods

In connection with the examples described herein, an exemplary device in accordance with the disclosed subject matter was fabricated from two patterned layers of poly(dimethylsiloxane) (PDMS; Sylgard 184; Dow-Corning) bonded to each other and sealed against a glass substrate (e.g., as depicted in FIG. 11A). The two PDMS layers were cast or double-cast from templates originally generated using standard photolithography of SU-8 on silicon wafers. Dimensions of certain features in both layers are shown in FIG. 11A. To assemble the device, the bottom layer was first sealed to a glass coverslip. The top and bottom layers were then treated with oxygen plasma, bonded together, and cured at 110° C. overnight. Assembled devices then were treated with oxygen plasma, immersed in 0.1 mg/ml poly-L-lysine (Sigma) for 1 hr, 1% glutaraldehyde (Sigma) for 1.5 hr, washed several times with ddH₂O, sterilized with UV light for 15 min, and soaked in 70% ethanol for 1 hr. To mold cylindrical channels, two 400 μm diameter acupuncture needles (Hwato) were inserted into parallel grooves at the top of the bottom layer and through the middle rectangular chamber approximately 200 μm above the glass coverslip surface. Rat tail collagen type I (2.5 mg/ml; BD Biosciences) was pipetted into the middle chamber and allowed to polymerize at 37° C. for 30 min. Excess collagen was subsequently aspirated from the fluid reservoirs feeding from the middle chamber. Devices were then covered with EGM-2 (Lonza) before the needles were extracted as described herein.

Cells were cultured and seeded in accordance with the disclosed subject matter into the device described in connection with the first example. Human umbilical vein endothelial cells (HUVECs) (Lonza) and human microvascular endothelial cells (HMVECs) (Lonza) were cultured in EGM-2 and EGM-2MV, respectively. While all experiments shown were conducted with HUVECs, HMVECs also sprouted in response to angiogenic cocktails. ECs were concentrated at 10⁷ cells/mL and seeded into one of the two channels. The device was inverted to allow ECs to adhere to the top surface of the channel for 10 min, and then flipped upright to allow cells to adhere to the bottom surface of the channel for another 10 min. Cells that adhered in the fluid reservoirs were scraped off with a pipette tip, and unattached cells in the channel were thoroughly flushed out with phosphate-buffered saline (PBS). Media was immediately added thereafter and the devices were placed on a platform rocker (BenchRocker, BR2000). Cells were cultured in channels for 1-2 days before angiogenic factors were introduced.

For immunofluorescence staining, cells in the devices were fixed in situ with 3.7% formaldehyde for 45 min. For CD31 labeling, cells were permeabilized with 0.1% Triton-X for 30 minutes, blocked in 3% BSA overnight at 4° C., washed 3 times with PBS and incubated with mouse monoclonal antibody against human CD31 (1:200, Dako). For laminin and podocalyxin labeling, samples were blocked with 3% BSA overnight at 4° C., washed 3 times with PBS and incubated with either rabbit polyclonal antibody against laminin (1:100, Chemicon) or goat polyclonal anti-human podocalyxin (1:100, R&D) overnight at 4° C. Before secondary antibody incubation, the devices were washed overnight with PBS at 4° C. All secondary antibodies (Invitrogen) were used at 1:500 dilution. Cell nuclei were labeled with DAPI (1:500, Sigma). F-actin was labeled with Alexa Fluor 488-conjugated Phalloidin (1:100, Sigma).

Brightfield images of sprouts, as described herein below, were acquired with a Nikon TE200 epifluorescence microscope (Nikon Instruments, Inc.) using 10× objective. Confocal immunofluorescence images were acquired with either 10× air objective or LD C-Apochromat 40x, 1.1 numerical aperture (N.A.) water immersion objective attached to either an Axiovert 200M inverted microscope (Zeiss) equipped with an CSU10 spinning disk confocal scan head (Yokogawa Electric Corporation), and an Evolve EMCCD camera (Photometrics) or an Olympus IX 81 microscope (Olympus America, Inc.) equipped with an CSU-X1 spinning disk confocal scan head (Yokogawa Electric Corporation), and an Andor iXon3 897 EMCCD camera (Andor Technology). ImageJ was used to merge channels, perform z-projection for all confocal stacks, and generate longitudinal and transverse cross-sections.

In connection with screening, the endothelialized parent vessel was perfused with culture media while the source channel was perfused with media enriched with angiogenic factors. Angiogenic factors include vascular endothelial growth factor (VEGF), monocyte chemotactic protein-1 (MCP-1), hepatocyte growth factor (HGF), and basic fibroblast growth factor (bFGF), all purchased from R&D Systems. Sphingosine-1-phosphate (S1P) and phorbol myristate acetate (PMA) were purchased from Cayman Chemical and Sigma, respectively. VEGF, MCP-1, bFGF, HGF, and PMA were all used at 75 ng/mL while S1P was used at 500 nM. Inhibitors targeting VEGFR2 (10 μM Semaxanib, Cayman Chemical) or S1P receptors (100 nM Fingolimod, Selleck Chemicals) were administered into both channels. MMP inhibitor (0.6 μM Marimastat, Tocris Bioscience) was administered into the source channel. Media in both channels were refreshed daily.

After neovessels bridged the two preformed channels in the device, a solution of CellTracker CM-DiI (Invitrogen) was delivered into the parent vessel to label cells in situ. Fluorescent beads (Polysciences) of 3 μm diameter were suspended in PBS and perfused into the parent vessel at a flow rate of 5 μL/min. Images were acquired at 40 frames/sec using an Eclipse TE2000 and an Evolve EMCCD camera.

Custom MATLAB code was written to measure the individual distances from the leading protrusions of tip cells to the wall of the parent vessel. Tip cells were additionally quantified as either attached to stalk cells extending from the endothelialized channel or as isolated single cells (FIG. S1). Sprouting metrics were quantified for the screening experiment (N=2 samples per condition), the VEGFR2 and S1P inhibitor experiment (N=5 samples per condition), and the MMPs inhibitor experiment (N=3 samples per condition).

Projections from z-resolved confocal stacks, which were taken with a 25× objective, Axiovert 200M inverted microscope (Zeiss), and spinning disk confocal scan head, were used to analyze filopodia length and number. A custom MATLAB code was used to determine the distance from the tips of filopodia to the center of cell nuclei and count the number of filopodia. The number and length of filopodia were averaged over the number of cells across 3 samples per condition.

Sample populations were compared using unpaired, two-tailed Student's t-test. P<0.05 was the threshold for statistical significance. Data points on the graphs represent mean values and error bars depict SEM.

Although the disclosed subject matter has been described in connection with particular embodiments thereof, it is to be understood that such embodiments are susceptible of modification and variations without departing from the inventive concept disclosed. All such modifications and variations, therefore, are intended to be included within the spirit and scope of the appended claims.

1. A device for 3D cell culture using at least one extracellular matrix, comprising:
a) a substrate having at least one interior chamber and at least one opening providing access to the at least one chamber for introduction of the at least one extracellular matrix into the chamber; and
b) at least one channel, disposed through at least a portion of the extracellular matrix for introduction of a fluid therethrough.

2. The device of claim 1, further comprising:
a) at least one reservoir fluidically coupled to the at least one channel for introduction of a fluid into the channel.

3. The device of claim 2, wherein the fluid is one of the group consisting of culture media, environment fluids, or biological fluids of the group of blood or its components, urine, milk, mucus, gastrointestinal fluids and bile.

4. The device of claim 2, wherein the fluid is a gas.

5. The device of claim 2, wherein the substrate further comprises:
a) a top layer having defined therein the at least one opening and at least one reservoir; and
b) a bottom layer having defined therein the at least one interior chamber and the at least one reservoir, whereby the at least one opening of the top layer is aligned with the at least one interior chamber, and the at least one reservoir of the top layer is aligned with the at least one reservoir of the bottom layer.

6. The device of claim 1, wherein the substrate comprises a PDMS substrate.

7. The device of claim 1, wherein the substrate is further positioned on a glass coverslip.

8. The device of claim 1, wherein the channel comprises a tubular channel.

9. The device of claim 2, wherein the at least one reservoir further comprises:
a) a first reservoir in fluid communication with a first end of the at least one channel; and
b) a second reservoir in fluid communication with a second end of the at least one channel.

10. The device of claim 2, wherein:
a) the at least one channel further comprises a plurality of channels; and
b) the at least one reservoir comprises a plurality of reservoirs, each channel in fluid communication with two reservoirs, each disposed on opposite ends of the channel.

11. The device of claim 10, wherein the at least one inner chamber comprises a single inner chamber.

12. The device of claim 2, wherein the media further comprises one of the group of any cell type, any type of fluid, or combination thereof.

13. A method of fabricating a device for 3D cell culture, comprising:
a) providing a top master mold for fabricating a top layer, the mold having raised portions defining at least one opening for introduction of extracellular matrix and at least one reservoir for introduction of media;
b) casting a top layer from the top master mold;
c) providing a bottom master mold for fabricating a bottom layer, the mold having raised portions defining an interior chamber and reservoirs for introduction of media, the reservoirs being connected to the interior chamber.
d) casting a bottom layer from the bottom master mold; and
e) treating the top layer and the bottom layer whereby the layers are adhered together.

14. The method of claim 13, further comprising:
a) inserting at least one object through the at least one microfabricated gap into the at least one interior chamber;
b) introducing a soluble extracellular matrix through the at least one opening into the at least one interior chamber, thereby encapsulating the at least one object; and
c) removing the at least one object after gelation of the extracellular matrix is complete, thereby defining at least one channel through the extracellular matrix.

15. The method of claim 14, further comprising:
a) sealing the microfabricated gaps.

16. A method for culturing cells using the device of claim 1, wherein the at least one channel comprises at least a first channel and a second channel, comprising:
a) introducing one or more cells in the first channel; and
b) introducing one or more of the group consisting of angiogenic factors, tumor cells, or an amount of one of the group of chemokines, cytokines, metabolites, toxins, and pharmacological compounds into the second channel.

17. The method of 16, wherein introduction of an angiogenic factor thereby creates a gradient extending across the extracellular matrix.

18. The method of claim 16, further comprising:
a) introducing immune cells into either the first channel after the endothelial cells have vascularized or into the extracellular matrix.

19. The method of claim 16, further comprising:
a) introducing one or more of the group consisting of immune cells, fibroblasts, chondrocytes, adipocytes, tumor cells, or stromal cells into the extracellular matrix.

20. The method of claim 16, wherein the cells introduced into the first channel are cells from tissues selected from the group consisting of brain, vasculature, pancreas, liver, gall bladder, spleen, intestine, mouth, nasopharynx, esophagus, peritoneal cavity, lung, trachea, kidney, bladder, ureter, prostate, and mammary gland.

21. The method of claim 16, further comprising:
a) varying perfusion characteristics of the media in the at least one reservoir, wherein said varied characteristics can be one of the group of direction, rate, and pressure.

22. The method of claim 16, further comprising:
a) using the at least first channel and second channel to generate intratubular and interstitial flow to provide increased transport to support interstitial tissue; and
b) studying the effects of flow and shear stress on cellular processes.

23. The method of claim 16, wherein the cells introduced into the first channel are patient derived cells.

24. The method of claim 23, further comprising:
a) observing the effects of one or more of the group consisting of angiogenic factors, tumor cells or an amount of one of the group of chemokines, cytokines, metabolites, toxins, and pharmacological compounds on the patient derived cells; and
b) identifying one or more of the group consisting of angiogenic factors, tumor cells or an amount of one of the group of chemokines, cytokines, metabolites, toxins, and pharmacological compounds as an effective drug or treatment for a particular patient based on the effects on the patient derived cells.

25. The method of claim 16, wherein the fluids are patient derived fluids.

26. A method for screening a compound for the ability to induce biological phenomena using the device of claim 1, wherein the at least one channel comprises at least a first channel and a second channel, comprising:
a) introducing one or more cells into the first channel;
b) selecting at least one compound for causing cells in the first channel to undergo one of the group consisting of singular and collective cell migration, angiogenic sprouting, vascular permeability, inflammatory cell invasion and migration, cancer cell invasion, extravasation, and migration, mammary cell sprouting and expansion, mammary cell milk production, pancreatic enzyme response, pancreatic cell survival, production of metabolic factors, bile production, renal filtration function, urinary production, pharyngeal-mucosal-gastrointestinal production of mucus, barrier function, and infectivity of the cells by viruses; and
c) introducing the selected at least one compound into a portion of the device.

27. The method of claim 26, further comprising:
a) observing an effect on the cells introduced into the first channel, thereby screening the selected compound.

28. The method of claim 27, wherein the compound is selected from the group consisting of: a gene, protein, siRNA, and small molecule.

29. A method for culturing cells to mimic tissue with coexisting tubular networks within at least one extracellular matrix using the device of claim 1, wherein the at least one channel comprises a plurality of channels, comprising:
a) introducing one or more cells of at least a first cell type in at least a first channel;
b) introducing one or more cells of at least a second cell type in at least a second channel; and
c) introducing one or more cell types into the extracellular matrix.
